(19) United States
(12) Patent Application Publication
(10) Pub. No.: US 2025/0232871 A1
(43) Pub. Date: Jul. 17, 2025

(54) PHYSIOLOGICAL TEST CREDIT METHOD

(71) Applicant: Willow Laboratories, Inc., Irvine, CA (US)

(72) Inventors: Marcelo M. Lamego, Coto De Caza, CA (US); Jeroen Poeze, Mission Viejo, CA (US)

(21) Appl. No.: 19/019,130

(22) Filed: Jan. 13, 2025

Related U.S. Application Data
(63) Continuation of application No. 17/741,315, filed on May 10, 2022, now Pat. No. 12,230,391.
Application No. 17/741,315 is a continuation of application No. 17/125,713, filed on Dec. 17, 2020, now Pat. No. 11,367,529.
Application No. 17/125,713 is a continuation of application No. 16/383,380, filed on Apr. 12, 2019, now abandoned.
Application No. 16/383,380 is a continuation of application No. 15/713,275, filed on Sep. 22, 2017, now Pat. No. 10,305,775.
Application No. 15/713,275 is a continuation of application No. 14/071,447, filed on Nov. 4, 2013, now Pat. No. 9,787,568.
(60) Provisional application No. 61/722,245, filed on Nov. 5, 2012.

(51) Int. Cl. G16H 40/67; A61B 5/00; A61B 5/1455; G06Q 20/14; H04L 9/32; H04L 43/16

(52) U.S. Cl. CPC G16H 40/67; A61B 5/0002; G06Q 20/145; H04L 9/3271; H04L 43/16; A61B 5/14551; A61B 2560/028

(57) ABSTRACT

A physiological test credit method determines if test credits are available to the monitor and checks if a Wi-Fi connection is available. If test credits are less than a test credit threshold, the monitor connects to a test credit server, processes server commands so as to download test credits and disconnects from the server. In various embodiments, the monitor is challenged to break a server code, the server is challenged to break a monitor code. The server validates monitor serial codes, and saves monitor configuration parameters.

### PRIORITY CLAIM AND REFERENCE TO RELATED APPLICATIONS

The present application is a continuation of U.S. patent application Ser. No. 17/741,315, filed May 10, 2022, titled Physiological Test Credit Method, which is a continuation of U.S. patent application Ser. No. 17/125,713, filed Dec. 17, 2020, titled Physiological Test Credit Method, which is a continuation of U.S. patent application Ser. No. 16/383,380, filed Apr. 12, 2019, titled Physiological Test Credit Method, which is a continuation of U.S. patent application Ser. No. 15/713,275, filed Sep. 22, 2017, titled Physiological Test Credit Method, which is a continuation of U.S. patent application Ser. No. 14/071,447, filed Nov. 4, 2013, now U.S. Pat. No. 9,787,568, titled Physiological Test Credit Method, which claims priority benefit under 35 U.S.C. § 119(e) to U.S. Provisional Patent Application Ser. No. 61/722,245, filed Nov. 5, 2012, titled Physiological Test Credit System, the entirety of each of which is hereby incorporated by reference herein.

### BACKGROUND OF THE INVENTION

Pulse oximetry is a widely accepted noninvasive procedure for measuring the oxygen saturation level of arterial blood, an indicator of a person's oxygen supply. A typical pulse oximetry system utilizes a sensor applied to a patient tissue site. The sensor has emitters that transmit optical radiation having red and infrared (IR) wavelengths into the tissue site. A detector responds to the intensity of the optical radiation after attenuation by pulsatile arterial blood flowing within the tissue site. Based on this response, a processor determines measurements for oxygen saturation and pulse rate. In addition, a pulse oximeter may display a plethysmograph waveform, which is a visualization of blood volume change within the illuminated tissue caused by the pulsatile arterial blood flow over time.

Pulse oximeters capable of reading through motion induced noise are available from Masimo Corporation (“Masimo”) Irvine, California. Moreover, portable and other pulse oximeters capable of reading through motion induced noise are disclosed in at least U.S. Pat. Nos. 6,770,028, 6,658,276, 6,157,850, 6,002,952 5,769,785, and 5,758,644, which are all assigned to Masimo and are all incorporated in their entireties by reference herein. Corresponding low noise sensors are also available from Masimo and are disclosed in at least U.S. Pat. Nos. 6,985,764, 6,813,511, 6,792,300, 6,256,523, 6,088,607, 5,782,757 and 5,638,818, which are all assigned to Masimo and are all incorporated in their entireties by reference herein. Such reading through motion pulse oximeters and low noise sensors have gained rapid acceptance in a wide variety of medical applications, including surgical wards, intensive care and neonatal units, general wards, home care, physical training, and virtually all types of monitoring scenarios.

Noninvasive blood parameter monitors capable of measuring blood parameters in addition to SPO₂, such as HbCO, HbMet and total hemoglobin (Hbt) and corresponding multiple wavelength optical sensors are also available from Masimo. Noninvasive blood parameter monitors and corresponding multiple wavelength optical sensors are described in at least U.S. patent application Ser. No. 11/367,013, filed Mar. 1, 2006 and entitled Multiple Wavelength Sensor Emitters and U.S. patent application Ser. No. 11/366,208, filed Mar. 1, 2006 and entitled Noninvasive Multi-Parameter Patient Monitor, both assigned to Cercacor Laboratories, Inc. (“Cercacor”) Irvine, CA and both incorporated in their entireties by reference herein.

Further, physiological monitoring systems that include low noise optical sensors and pulse oximetry monitors, such as any of LNOP® adhesive or reusable sensors, SofTouch™ sensors, Hi-Fi Trauma™ or Blue™ sensors; and any of Radical®, SatShare™, Rad-9™, Rad-5™, Rad-5v™ or PPO+™ Masimo SET® pulse oximeters, are all available from Masimo. Physiological monitoring systems including multiple wavelength sensors and corresponding noninvasive blood parameter monitors, such as Rainbow™ adhesive and reusable sensors and RAD-57™ and Radical-7™ monitors for measuring SpO₂, pulse rate, perfusion index, signal quality, HbCO and HbMet among other parameters are also available from Masimo.

### SUMMARY OF THE INVENTION

A physiological monitoring system has a sensor that transmits optical radiation at a multiplicity of wavelengths and a monitor that determines the relative concentrations of blood constituents such as HbO₂, Hb, carboxyhemoglobin (HbCO), methemoglobin (MetHb), fractional oxygen saturation, total hemoglobin (Hbt) and blood glucose to name a few. The monitor advantageously utilizes test credits each of which represent a quantum of currency. A test credit enables the monitor to make a physiological measurement. In various embodiments different monitor buttons are pressed to measure different variables. For example, one button initiates a SpO₂ measurement and another button initiates a SpCO measurement. After the measurement, the number of available test credits are decremented. The sensor has a memory that stores the test credits, which is decremented after each test. The monitor reads the sensor memory so as to determine the remaining test credits. Physiological monitoring systems include a Masimo Pronto-7® and corresponding rainbow 4D™ DC sensor. The Pronto-7 is a palm-sized device designed for quick-and-easy noninvasive total hemoglobin (SpHb®) spot-check testing, along with SPO₂, pulse rate, and perfusion index. A spot check monitor and corresponding credit system is described in U.S. patent application Ser. No. 12/882,111 titled Spot Check Monitor Credit System, filed Sep. 14, 2010 and U.S. patent application Ser. No. 13/110,833 titled Spot Check Monitor Credit System, filed May 18, 2011, both assigned to Cercacor and both incorporated in their entireties by reference herein.

An aspect of a physiological test credit method programmatically initiates wireless communications between a physiological monitor and a remote server in response to available test credits falling below a predetermined threshold so as to download additional test credits from the server to the monitor and therefore enable the monitor to perform additional physiological parameter spot-checks on a per test credit basis. The physiological test credit system establishes a threshold for test credits stored in an optical sensor attached to a corresponding physiological monitor. A server is securely connected to the monitor when remaining test credits fall below the threshold, and test credits are downloaded from the server to the monitor accordingly. In various embodiments, a quantum of test credits is defined and an amount of test credits equal to the quantum is downloaded each time the remaining test credits fall below the threshold. A download frequency is defined according to the number of times remaining test credits fall below the threshold in a given period of time. The threshold is adjusted according to the download frequency. Alternatively, or in addition to adjusting the threshold, the quantum is adjusted according to the download frequency.

In various other embodiments, challenges are exchanged between the server and the monitor so as to verify both the server and the monitor. The challenges may include sending a server challenge code to the monitor and breaking the server challenge code with the monitor and sending a monitor challenge code to the server and breaking the monitor challenge code with the server. The monitor may be validated utilizing sales data. Also, the server may send a request for device serial numbers to the monitor, and the serial numbers are matched to sales data. Further, monitor validation may include sending a server request for a zip code to the monitor and matching the zip code to the monitor account.

Another aspect a physiological test credit method is determining a test credit quantity associated with a physiological sensor, comparing the test credit quantity to a threshold and wirelessly connecting a physiological monitor to a server if the test credit quantity is below the threshold. Server commands are processed so as to download additional test credits to the monitor. Additional test credits are transferred to the physiological sensor, and the physiological monitor is disconnected from the server.

In various embodiments, the frequency of test credit downloads are tracked and, at least periodically, the amount of additional test credits downloaded are adjusted according to the frequency. The threshold may be adjusted, at least periodically, according to the frequency. The monitor may be challenged to break a server code before any test credits are downloaded to the monitor. The server may be challenged to break a monitor code before any test credits are downloaded to the monitor.

A further aspect of a physiological test credit method comprises establishing a wireless connection between a monitor and a server; downloading a file of test credits from the server to the monitor; transferring the test credits to a sensor in communications with the monitor; performing a physiological test on a person using the sensor; displaying a result of the physiological test on the monitor; deducting a test credit from the sensor in response to the test; and downloading an additional file of test credits from the server to the monitor in response to the number of test credits remaining in the sensor falling below a threshold.

In various embodiments, a quantity of test credits contained in the additional file are defined and adjusted according to the time between the downloading of the file and the downloading of the additional file. The threshold is adjusted according to the time between the downloading of the file and the downloading of the additional file. A two-way challenge is performed between the monitor and the server before downloading a file of test credits. The two-way challenge comprises sending a server challenge code to the monitor; breaking the server challenge code at the monitor; sending a monitor challenge code to the server; and breaking the monitor challenge code at the server. The sensor and the monitor serial numbers are sent to the server, and the serial numbers are matched with sales data available to the server. Monitor configuration parameters are saved on the server.

Yet another aspect of a physiological test credit system establishes wireless communications between a physiological monitor and a remote server. The server downloads test credits from the server to a sensor attached to the monitor. Each test credit allows the monitor to perform a spot-check of a physiological parameter. The monitor initiates a test credit download when the number of available test credits for a sensor falls below a test credit threshold. The monitor establishes a wireless connection with a server. The monitor and server perform a two-way challenge so that each can verify a connection to an approved device. The server also authenticates the monitor's account on the server. If the challenges and authentication are successful, the server downloads a credit file of test credits to the monitor and its attached sensor. The monitor and server then disconnect.

An additional aspect of a physiological test credit method establishes wireless communications between a physiological monitor and a remote server so as to download test credits to the monitor and its attached optical sensor. This enables the monitor to perform physiological parameter spot-checks on a per test credit basis. The physiological test credit method establishes a minimum threshold for test credits stored in an optical sensor attached to a corresponding physiological monitor and connects a server to the monitor when remaining test credits fall below the minimum threshold. The server-monitor connection first requires the monitor and server to exchange mutual code-breaking challenges for server-monitor verification. The server is then allowed to authenticate the monitor user account and funds. The server further validates the monitor via sales data. Monitor settings are saved to the server, and server commands are sent to the monitor.

In various embodiments, a server command is sent to the monitor to download configuration parameters including the test credit minimum threshold. The first time a monitor is connected to a server, a one-time monitor setup is performed. In exchanging challenges a server challenge code is sent to the monitor. The monitor must break the server challenge code. Then the monitor sends a challenge code to the server, which the server must break. Monitor validation involves a server request for device serial numbers from the monitor, which are matched with sales data. The server also requests a monitor zip code, which is matched with the monitor account. In various other embodiments, user settings are saved in the monitor and downloaded to the server. The server sends monitor commands that comprise OP commands to perform a monitor functions, GUI commands to display results and hybrid command to initiate a user response.

A further aspect of a physiological test credit method comprises establishing a wireless connection between a monitor and a server, performing a two-way challenge between the monitor and the server, authenticating the monitor and validating the monitor. The method may further comprise transferring a credit file of test credits from the server to the monitor, performing a physiological test, deducting a test credit corresponding to the test from the monitor account, and disconnecting the monitor and the server. The server also performs a one-time monitor setup.

In various embodiments, the two-way challenge includes sending a first challenge code from the server to the monitor, breaking the first challenge code at the monitor, sending a second challenge code from the monitor to the server and breaking the second challenge code at the server. A monitor validation comprises sending sensor and monitor serial numbers to the server, matching the serial numbers with sales data available to the server, sending a zip code to the server and matching the zip code with account information available to the server. Authenticating may comprise verifying the monitor's user account and funds. Setting-up the monitor may comprise sending commands and queries to the monitor so as to receive configuration parameters.

Another aspect of a physiological test credit method determines if test credits are available to the monitor and checks if a Wi-Fi connection is available. If test credits are less than a test credit threshold, the monitor connects to a test credit server, processes server commands so as to download test credits and disconnects from the server. In various embodiments, the monitor is challenged to break a server code, the server is challenged to break a monitor code. The server validates monitor serial codes, and saves monitor configuration parameters.

### BRIEF DESCRIPTION OF THE DRAWINGS

FIG. 1 is a flowchart of a monitor-side physiological test credit method;

FIGS. 2-3 are flowcharts of a server-side physiological test credit method;

FIG. 4 is a flowchart of a two-way challenge for both the server and the monitor to verify their connections;

FIG. 5 is a flowchart of server and monitor account validation;

FIG. 6 is a flowchart of a server one-time monitor setup;

FIG. 7 is a flowchart of server command and monitor acknowledgement communications; and

FIGS. 8A-C are flowcharts of server communications of and monitor responses to operational, GUI and hybrid commands.

### DETAILED DESCRIPTION OF THE PREFERRED EMBODIMENTS

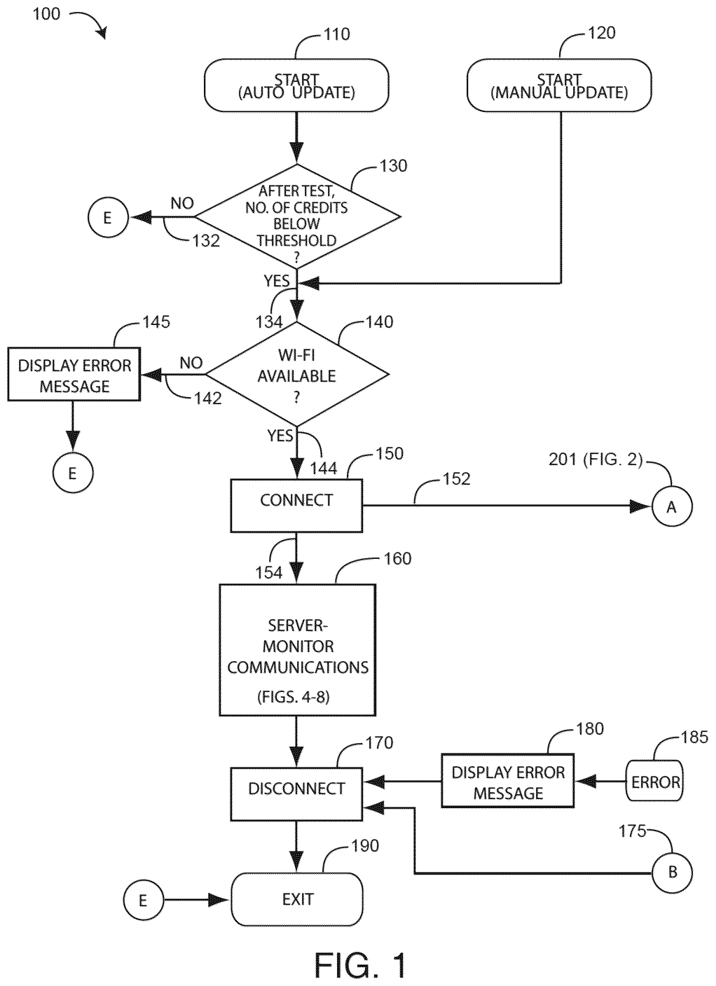

FIGS. 1-3 illustrate test credit downloads to a monitoring device using a broadband connection, such as Wi-Fi. In an embodiment, the monitor always initiates the connection. There are two ways a user can download test credits. “Automatic download” or “Manual download.” For automatic downloads, the monitor initiates the connection after a test is performed and the available test credits fall below a predefined update threshold. For manual downloads, a user initiates the connection using a monitor menu flow. Accordingly, when a monitor initiates a connection, the initial connection request will have a parameter that describes whether or not a user manually initiated the connection.

For an automatic download, the monitor initiates a connection to a test credit server after a test is performed and the test credits drop below a user-predefined update threshold. In an embodiment, a user-predefined quantum of test credits (an integer between 1 and N inclusive) is downloaded each time the update threshold is crossed. In an embodiment, the test credit update threshold and the test credit download quantum are user-defined. In an embodiment, the monitor automatically adjusts the test credit download quantum and/or the test credit update threshold according to the frequency of test credit usage so as to advantageously reduce the frequency of server connections during periods of heavy monitor usage and to advantageously spread-out test credit expenditures during periods of light monitor usage. In an embodiment, the relationship between test credit download quantum, test credit update threshold and test credit usage is user defined.

The process of updating the test credits is done in the background. However, a user will not be able to perform a test during this period. If insufficient test credits are available for auto-download, a server may query/inform a user about this scenario via GUI commands to the monitor. A monitor initiates a manual download via a local GUI. Then the server takes over the process as with an auto download mode.

FIG. 1 illustrates a test credit download process from a monitor perspective 100. A monitor test credit download can be initiated automatically 110. After a test is complete, the number of available tests credits on a connected sensor is determined and compared with a specified update threshold 130. If the available test credits have not fallen below that threshold 132, the update process exits 190. If the number of test credits are below that threshold 134, the update process continues.

Also shown in FIG. 1, alternatively, a monitor test credit download can be initiated manually when a user selects a manual test credit update menu option 120. For either auto update 110 or manual update 120, Wi-Fi availability is determined 140. If there is no Wi-Fi 142, the process displays an error message 145 and exits 190. If Wi-Fi is available 144, a connection 150 to a server is attempted 152, as described below. Once a server connection is established 154, the monitor processes commands from the server 160, disconnects 170 and exits 190.

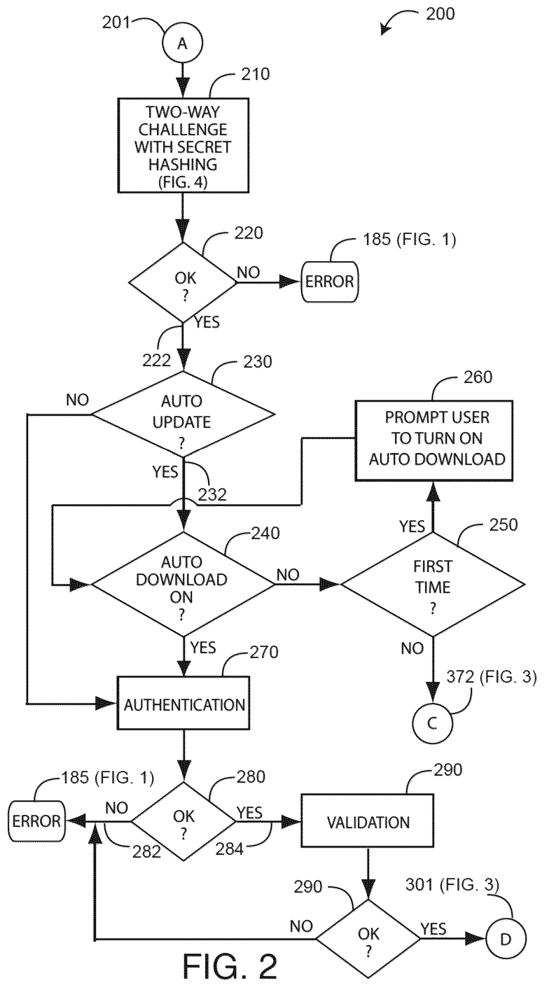

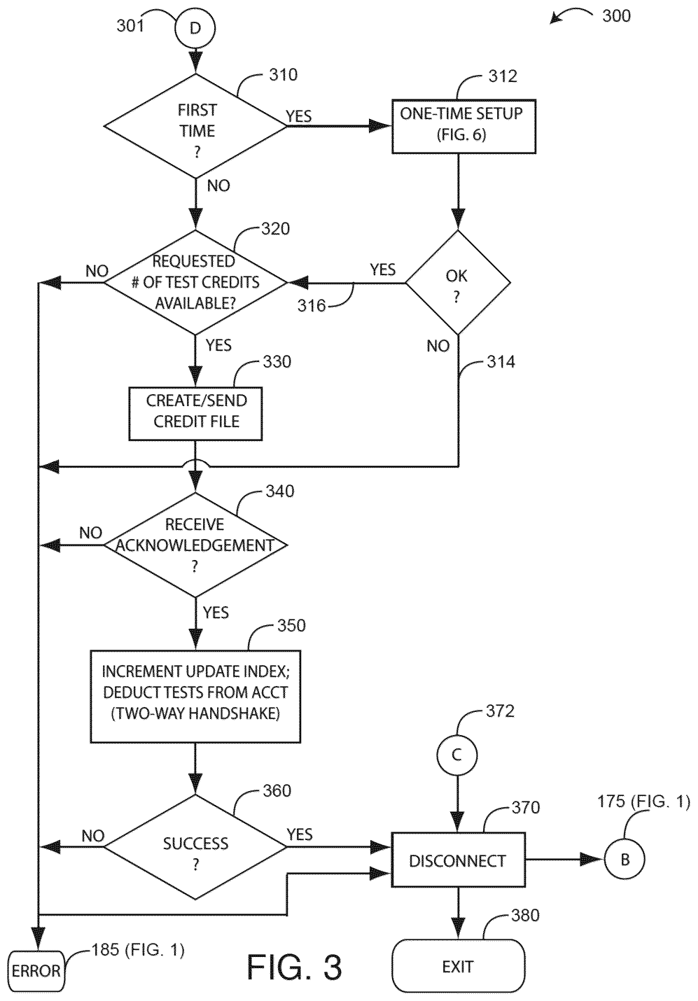

FIGS. 2-3 illustrate a test credit download process from a server perspective. As shown in FIG. 2, immediately after a Wi-Fi connection is established between monitor and server, the server initiates a two-way challenge with secret hashing 210. Both the server and the monitor use a two-way challenge to verify their connection with a real monitor or server, respectively, as described in detail with respect to FIG. 4, below. If the challenge 210 is successful 222, the server determines if the update is manual or automatic 230. If automatic 232, the server verifies that the user has turned on the auto download feature 240, prompting the user 250, 260 if necessary. If the auto download remains off, the server disconnects 372 (FIG. 3). With either the manual or automatic update, a secure authentication 270 is performed, which verifies the user's account and funds. If authentication fails 282, an error 185 (FIG. 1) is returned to the monitor, an error message is displayed 180 (FIG. 1) and the monitor disconnects 170 (FIG. 1). If authentication succeeds 284, validation is performed 301 (FIG. 3).

As shown in FIG. 2, once the user's account is authenticated, the customer and/or monitor and sensor are validated 290. In part, this step insures that customers, monitors and sensors are paired to the appropriate manufacturer or distributor. This can be a one-time validation or a validation for every credit download.

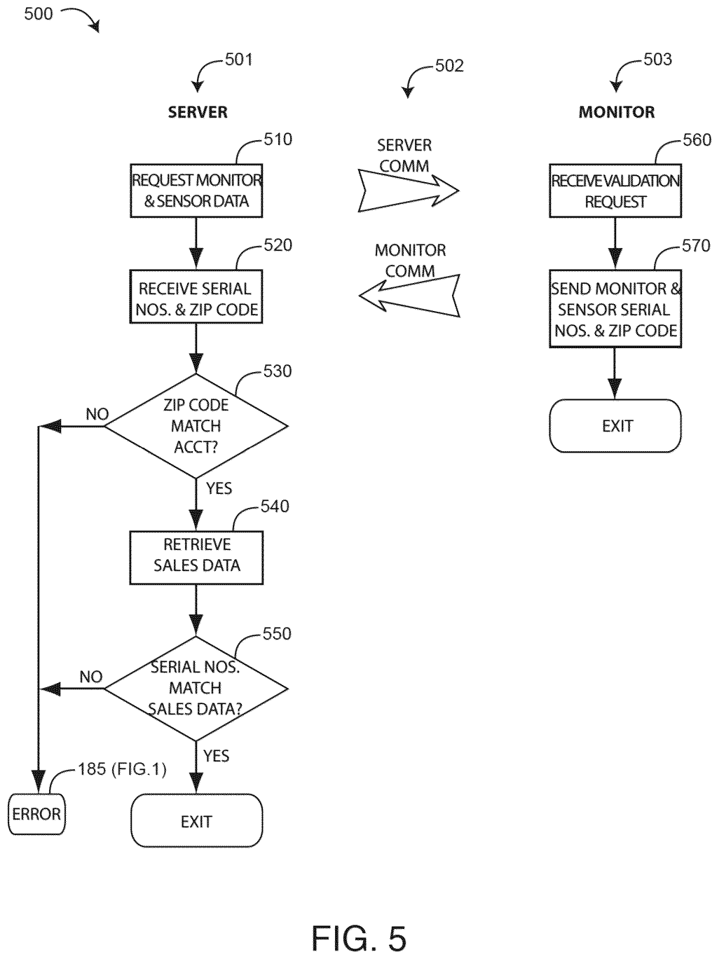

As shown in FIG. 3, when the server recognizes that a monitor is connecting for the first time 310, the server performs a one-time setup 312, as described with respect to FIG. 6, below. If there is any problems with the setup 314, the server sends an error 185 (FIG. 1) to the monitor, drops the connection 370 and exits 380. If the setup 312 is currently successful 316 or was previously performed 310, the server determines if the requested number of test credits are available 320. If so, the credit file is created and sent to the monitor 330. If not, an error is returned to the monitor 185 (FIG. 1), and the server disconnects 370 and exits 380.

Further shown in FIG. 3, after the monitor receives a credit file from the server 330. The monitor sends an ACK and the server receives the ACK 340. Using hand-shaking, either zero or all three of the following operations take place: the server deducts the credit from user account; the server increments the index count; and/or the monitor updates credits into the sensor 350.

Additionally shown in FIG. 3, the server drops the connection 370 upon successful completion of the process 360 or if it recognizes any error 185 (FIG. 1). The monitor drops the connection 170 (FIG. 1) if the server does not meet the monitor's challenge or the monitor recognizes an error 185 due to any reason.

FIG. 4 illustrates the two-way challenge 400 for both the server 401 and the monitor 403 to verify their connections. In order to do that, both the monitor and the server challenge each other with a code to break, which only a real server and a real monitor know how to do. In particular, the server 410 first challenges the monitor 450 to break a code. If the server sees that the device cannot break the code 420, the server drops the connection. Otherwise, the server 430 asks the monitor 470 to send the server a code to break. If the monitor sees that the server cannot break the code 480, the monitor drops the connection.

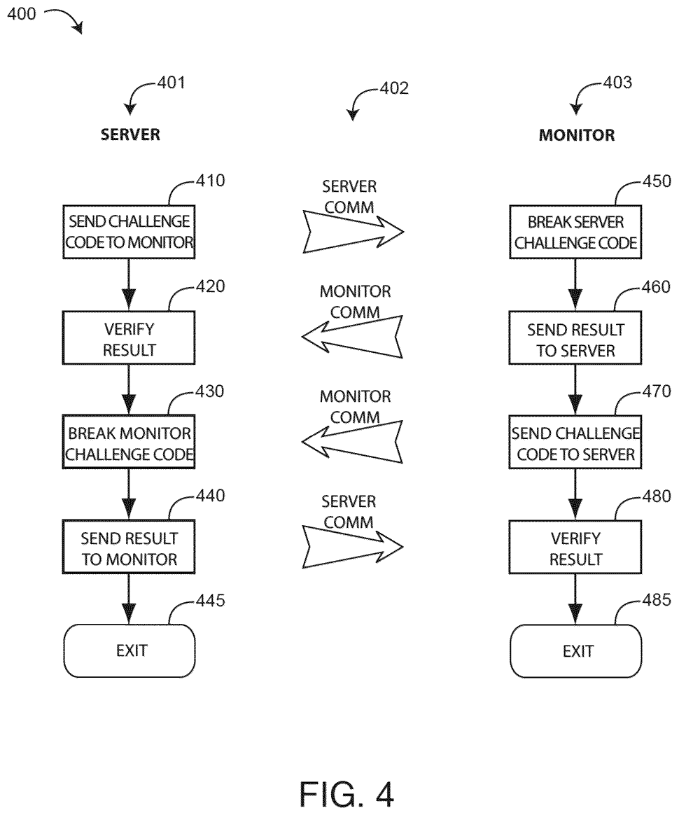

FIG. 5 illustrates monitor and user account validation 500. Validation is performed for direct from manufacturer sales or for sales through distributors. Several validation methods can be used. Monitor and a sensor serial numbers can be compared with sales order records or with tracing data from distributors. A zip code can be used in a manner similar to a credit card, i.e. to prevent a user from accessing a wrong account due to incorrect recording of serial numbers. An address can be used the same as a zip code, but is more secure. However, an address is prone to user error and free text input is difficult to validate. A web registration can be used for distribution sales or direct acute care sales.

As shown in FIG. 5, the server 501 requests monitor and sensor data 510. The monitor 503 receives the request 530 and complies 570. The server 501 receives the requested data 520, which is compared with server records 530-550 for a match. The server sends an error message 185 (FIG. 1) for a monitor to display 180 (FIG. 1), such as Contact Tech Support, if validation data does not match with sales records.

FIG. 6 illustrates a server 610 performing a monitor setup 600. The server 601 sends saved settings 610 to the monitor 630. The user can always change settings online 640. The monitor 650 sends these to the server to save 620. Any configuration parameters that the monitor needs to remember, such as the low credit threshold, are saved on the server side 601. The server 601 initially sends a set of commands/queries to the monitor to get configuration parameters (not shown). These parameters include: turn on/off auto-download; define threshold that initiates automatic download; and define number of credits to automatically download. The server may also perform an extra one-time validation, using, e.g., a zip code or the last 4 digits of an account number. If any errors occur on the monitor side, a local message is displayed and the connection is dropped. If any errors occur on server side, the server may choose to display an error message on the server side (via GUI commands) and drop the connection.

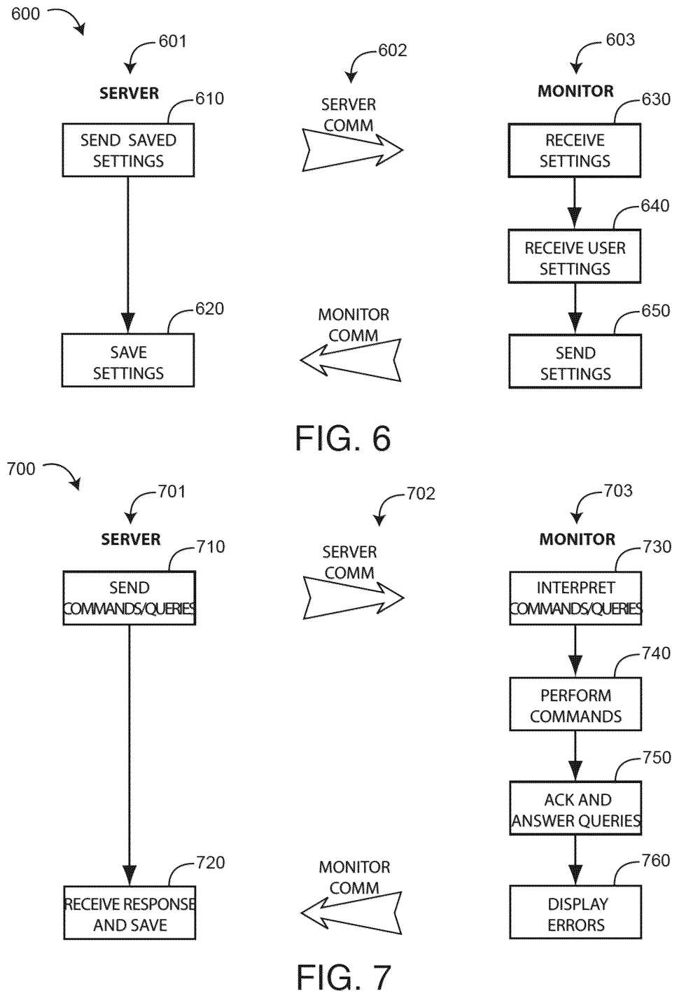

FIG. 7 illustrates server command and monitor acknowledgement communications 700. Once a connection is established, the server assumes the master role and the monitor assumes a slave role. The server 701 sends commands/queries 710, which the monitor 703 interprets 30. The monitor 703 performs the commands 740 and acknowledges/answers the queries 750. Any errors are displayed 760 on the monitor 703. The sensor 701 saves the monitor responses 720. In an embodiment, the server sends commands in a form of HTML-like NMEA data.

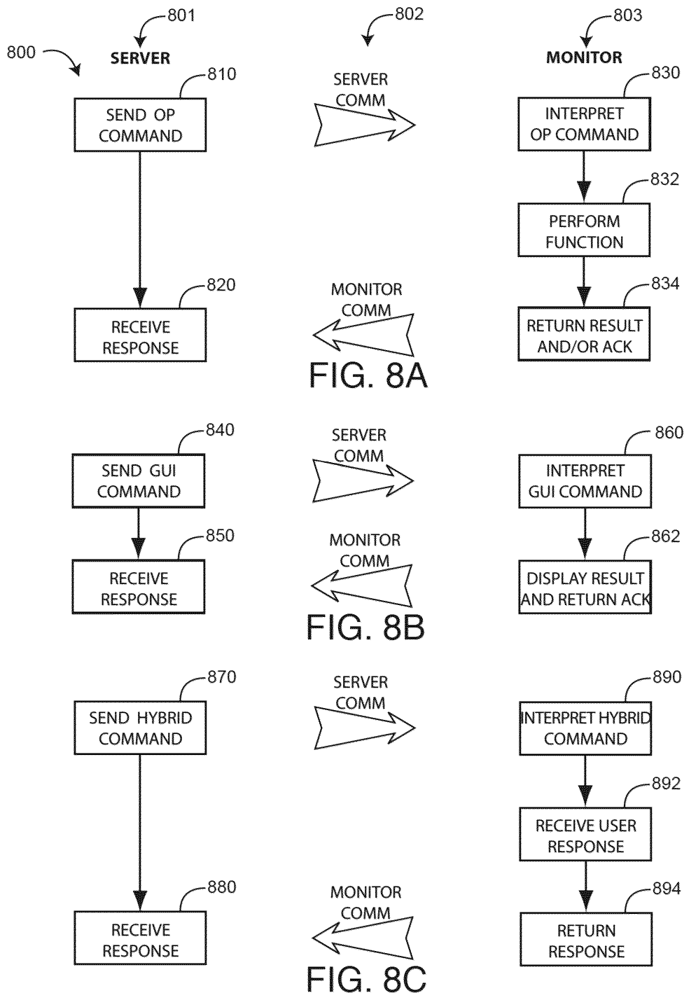

FIGS. 8A-C illustrate server commands and monitor responses to those commands. The server 801 may send three types of commands including an operational command 810, a GUI command 840 and a hybrid command 870. For example, an operational command 810 may be “get zip code,” or “accept credit file and send back ACK.” GUI commands 840 may be, for example, display message “online credit update in progress.” A hybrid command 870 may be to display a GUI as well as expect a response from the monitor user. An example would be a command to display the message “want to turn on auto update?” and wait for a “yes” or “no” response.

As shown in FIGS. 8A-C, the monitor 803 response to an op command 830 is to perform a function 832 and return a result and/or ACK 834. The monitor 803 response to a GUI command 860 is to display a result and ACK 862. The monitor 803 response to a hybrid command 890 is to receive and return a user response 892, 894.

A physiological test credit method has been disclosed in detail in connection with various embodiments. These embodiments are disclosed by way of examples only and are not to limit the scope of the claims that follow. One of ordinary skill in art will appreciate many variations and modifications.

1. (canceled)

2. A physiological monitoring system configured to perform parameter spot-checks on a per test credit basis, the system comprising:
a remote server comprising a processor and a memory, wherein the memory includes computer-executable instructions; wherein the processor of the remote server is configured to establish secure communication with a physiological monitor by:
receiving a monitor challenge code from a physiological monitor;
establishing a secure communication channel between the physiological monitor and the remote server in response to breaking the monitor challenge code;
sending test credits to the physiological monitor;
deduct test credit from a user account at the remote server;
increment a test credit downloading index count; and
cause, through the secure communication channel, an update to a test credit total saved to a memory of a sensor in communication with the physiological monitor.

3. The physiological monitoring system of claim 2, wherein the remote server is configured to send a server challenge code to the physiological monitor.

4. The physiological monitoring system of claim 3, wherein the remote server is further configured to drop the connection with the physiological monitor in response to the physiological monitor's inability to break the server challenge code.

5. The physiological monitoring system of claim 3, wherein the remote server is further configured to request the physiological monitor to send the monitor challenge code for the remote server to break in response to the physiological monitor breaking the server challenge code.

6. The physiological monitoring system of claim 3, wherein the server challenge code comprises secret hashing.

7. The physiological monitoring system of claim 2, wherein the remote server is configured to determine whether the update is manual or automatic.

8. The physiological monitoring system of claim 2, wherein the remote server is configured to perform the update by providing a credit file for the physiological monitor to download.

9. The physiological monitoring system of claim 2, wherein the remote server is further configured to validate a monitor or user account by requesting monitor and sensor data prior to performing the update.

10. The physiological monitoring system of claim 2, wherein the remote server acts in a master role and the physiological monitor acts in a slave role once the connection is established.

11. The physiological monitoring system of claim 2, wherein the remote server is configured to receive a request to establish the connection from the physiological monitor in response to the one or more test credits falling below a predefined update threshold.

12. A method to establish a connection to update one or more test credits used to perform parameter spot-checks on a per test credit basis, the method comprising:
receiving, at a remote server, a monitor challenge code from a physiological monitor;
establishing a secure communication channel between the physiological monitor and a processor of the remote server in response the remote server breaking the monitor challenge code;
sending test credit from to the physiological monitor;
deducting the test credit from a user account at the remote server;
incrementing a test credit downloading index count at the remote server; and
causing, through the secure communication channel, an update to a test credit total saved on the memory of a sensor in communication with the physiological monitor.

13. The method of claim 12, comprising sending a server challenge code from the remote server to the physiological monitor.

14. The method of claim 13, comprising dropping, using the remote server, the connection between the remote server and the physiological monitor in response to the physiological monitor's inability to break the server challenge code.

15. The method of claim 13, comprising requesting the physiological monitor to send the monitor challenge code for the remote server to break in response to the physiological monitor breaking the server challenge code.

16. The method of claim 13, wherein the server challenge code includes secret hashing.

17. The method of claim 12, comprising determining, using the remote server, whether the update is manual or automatic.

18. The method of claim 12, comprising performing, using the remote server, the update by sending a credit file to the physiological monitor.

19. The method of claim 12, comprising validating, using the remote server, a monitor or user account by requesting monitor and sensor data prior to performing the update.

20. The method of claim 12, comprising receiving, using the physiological monitor, a request to establish the connection with the remote server in response to the one or more test credits falling below a predefined update threshold.
